(19) United States
(12) Patent Application Publication
(10) Pub. No.: US 2023/0360769 A1
(43) Pub. Date: Nov. 9, 2023

(54) PROCESS FOR DETERMINING RISK FOR PSEUDOPHAKIC NEGATIVE AND POSITIVE DYSPHOTOPSIA

(71) Applicants: Michael Snyder, Cincinnati, OH (US); Nicole Fram, Los Angeles, CA (US)

(72) Inventors: Michael Snyder, Cincinnati, OH (US); Nicole Fram, Los Angeles, CA (US)

(21) Appl. No.: 18/312,966

(22) Filed: May 5, 2023

Related U.S. Application Data
(60) Provisional application No. 63/338,567, filed on May 5, 2022.

(51) Int. Cl. G16H 20/40; A61B 3/12; A61B 3/10; A61F 2/16

(52) U.S. Cl. CPC G16H 20/40; A61B 3/12; A61B 3/102; A61F 2/16; A61F 2002/1696

(57) ABSTRACT

A process for reducing the likelihood of or correcting dysphotopsia from intraocular lens implant surgery. The process includes a step of scanning the eye of a patient in need of the intraocular lens implant surgery using a noninvasive echo-locating instrument for obtaining nasal retina information about the eye. Based at least in part from the nasal retina information, a prophylactic measure can be recommended prior to cataract removal surgery, or a corrective measure can be recommended to address the dysphotopsia after the cataract removal surgery. Use of artificial intelligence as part of the process is also contemplated.

### CROSS-REFERENCE TO RELATED APPLICATIONS

This application claims priority to U.S. Patent Provisional Application No. 63/338,567 filed on May 5, 2022, the complete disclosure of which, in its entirety, is hereby incorporated by reference.

### FIELD

The present teachings generally relate to a process to determine, prior to cataract removal surgery, which eyes are at higher risk for development of complications from intraocular lens (“IOL”) implants, such as the occurrence of negative and/or positive dysphotopsia.

### BACKGROUND

Aphakia is an ophthalmic condition whereby a natural lens is absent from the eye. Aphakia can result from removal of the natural lens during cataract surgery, genetic disorders causing birth without a natural lens, or dislocation of a natural lens by an injury and/or genetic susceptibility to dislocation of a natural lens. Aphakia is typically associated with an impairment in the ability to focus on objects that the afflicted (patient) is viewing.

Aphakia can be treated by surgical implantation of an intraocular lens implant. In a phakic eye, the intact natural crystalline lens, otherwise referred to herein as a natural lens, has a convex anterior contour and the pupil glides along the anterior contoured surface as it expands or contracts. In a pseudophakic eye, the intraocular lens implant is thinner and has a lesser anterior convexity relative to a natural crystalline lens. The anterior surface of the intraocular lens implant thereby rests more posterior in the eye than that of the natural lens. The intraocular lens implant also has a smaller diameter relative to the natural crystalline lens. This pseudophakic anatomy results in a potential space between the posterior surface of the iris and the anterior surface of the intraocular lens implant after cataract surgery. In other words, the lesser thickness and positioning of the intraocular lens implant relative to the natural crystalline lens gives rise to the potential space. Light entering the eye along a path that is oblique (e.g., tangential) to the intraocular lens implant can pass through this space, particularly in a physiologically nasal region. Due to both the increased antero-posterior space and the relatively smaller intraocular lens implant diameter, some of this light may strike the edge of the intraocular lens implant and be refracted posteriorly toward the nasal retina while other light rays may be un-refracted and may strike the nasal retina more anteriorly than the refracted light rays. As a result, patients can have a dark area in the temporal area of their visual field resulting from the so-called “illumination gap” (Erie, Holladay) that is a narrow band of non-illuminated retina that is bounded in both sides by a brighter region. Holladay J T, Simpson M J. Negative dysphotopsia: causes and rationale for prevention and treatment. J Cataract Refract Surg 2017; 43:263-275; and Erie J C, Simpson M J, Bandhauer M H. A modified intraocular lens design to reduce negative dysphotopsia. J Cataract Refract Surg 2019 July; 45(7):1013-1019.

Some patients report of the dark area being crescent-shaped or line-shaped, although other shapes may be possible. This condition is conventionally referred to as negative dysphotopsia.

Positive dysphotopsia is perceived as light arcs, starburst, and halos stimulated by an oblique light source. The etiology is better understood than negative dysphotopsia and is a result of both diffraction and internal reflection from light rays hitting the edge of the intraocular lens. It is also associated with smaller optic size and high index of refraction. Holladay J T, Bishop J E, Lewis J W. Diagnosis and treatment of mysterious light streaks seen by patients following extracapsular cataract extraction. Am Intra-Ocular Implant Soc J 1985; 11:21-23; Masket S, Geraghty E, Crandall A S, Davison J A, Johnson S H, Koch D D, Lane S S. Undesired light images associated with ovoid intraocular lenses. J Cataract Refract Surg 1993; 19:690-694; and Holladay J T, Lang A, Portney V. Analysis of edge glare phenomena in intra-ocular lens edge designs. J Cataract Refract Surg 1999; 25:748-752.

Many patients notice dysphotopsias early on in their post-operative course, though in the majority of patients, the symptoms gradually abate over days-to-weeks or, occasionally, months. In some patients, the symptoms may fade due to physical healing factors. In other patients, the resolution of symptoms may be a neuroadaptive process. Intraocular lens implant edge designs may impact the quality and intensity of the visual phenomenon.

In addition to and a part of the intraocular lens implant positioning within the eye, the width and curvature of the cornea, the depth of the anterior chamber, the natural lens thickness, and the alignment of the eye relative to the optical axis (angle alpha or angle kappa, for example) have all been implicated as potential risk factors for dysphotopsia. Holladay J T, Zhao H, Reisin C R. Negative dysphotopsias: the enigmatic penumbra, J Cataract Refract Surg 2012; 38:1251-1265; and Makhotkina N Y, Nijkamp M D, Berendschot T T J M, van den Borne B, Nuijts R M M A. Effect of active evaluation on the detection of negative dysphotopsia after sequential cataract surgery: discrepancy between incidences of unsolicited and solicited complaints, Acta Ophthalmol 2018; 96:81-87.

Other theories have been advanced about causal factors associated with dysphotopsias and include, for example, IOL material, ring scotoma/high power intraocular lens, nasal capsule overlap/capsulorhexis, index of refraction, posterior chamber depth, biconvex intraocular lens, temporal corneal incision, displacement and/or magnification of a blind spot, and transversal propagation of light reflected internally within a lens. See, e.g., Osher R H. Negative dysphotopsia: long-term study and possible explanation for transient symptoms. J Cataract Refract Surg 2008; 34:1699-1707; Makhotkina N Y, Nijkamp M D, Berendschot T T J M, van den Borne B, Nuijts R M M A. Effect of active evaluation on the detection of negative dysphotopsia after sequential cataract surgery: discrepancy between incidences of unsolicited and solicited complaints, Acta Ophthalmol 2018; 96:81-87; and Masket S, Fram N R, Cho A, Park L, Pham D. Surgical management of negative dysphotopsia, J Cataract Refract Surg 2018; 44:6-16; and Makhotkina N Y, Berendschot T T J M, Nuijts R M M A. Objective evaluation of negative dysphotopsia with Goldmann kinetic perimetry. J Cataract Refract Surg 2016; 42:1626-1633.

Some solutions to these other ophthalmic conditions causing dysphotopsia have been proposed but they do not address light passing through a gap between the iris and intraocular implant lens. For instance, U.S. Patent Application Publication No. 2008/0269891 A1, incorporated by reference in its entirety, describes an intraocular lens implant with modified edge characteristics that inhibit transverse propagation of internally reflected light rays. Some investigators believe that the nasal capsule margin overlap of the intraocular lens may be a causative factor in some forms of negative dysphotopsia. Masket has patents addressing negative dysphotopsia and has developed an anti-dysphotopsia intraocular lens that captures the anterior capsulorhexis in a groove (U.S. Pat. Nos. 8,652,206 and 9,433,498; both incorporated by reference in their entirety). However, while these patents treat one possible cause of negative dysphotopsia, it does not address light passing through a gap between the iris and intraocular lens implant. With respect to addressing the gap, some have proposed implanting an intraocular lens implant in the ciliary sulcus rather than in the capsular bag. Others have proposed implanting an additional low or no powered clear intraocular lens implant in the ciliary sulcus while a prior implanted intraocular lens implant remains in the capsular bag. Snyder has taught that an ophthalmic prosthetic placed either within the capsular bag or ciliary sulcus could be an effective treatment for positive and negative dysphotopsias (U.S. patent application Ser. No. 17/405,741; incorporated by reference in its entirety). While any, some, none, or all of these potential treatments for dysphotopsias may be effective, it is believed that there is presently no systemic paradigm to identify “at risk” eyes a priori of cataract surgery. Identification of “at risk” eyes could lead to both better education of the patient of expectations and, perhaps more importantly, identification of individuals (or individual eyes) for prophylactic treatment(s) of dysphotopsias.

### SUMMARY

The present teachings are predicated, at least in part, upon a recognition that a substantial contributing factor toward a heightened risk of dysphotopsia risk, and especially for negative dysphotopsia, is the location of the anterior extent of the nasal retina. Accordingly, in one aspect, the teachings herein envision that the nasal retina is analyzed and information from the analysis is used for predicting the likelihood of dysphotopsia before cataract removal surgery is performed upon a patient in need thereof.

It is also contemplated as within the teachings that, following a cataract removal surgery performed upon a patient in need thereof and after implantation of an intraocular lens (“IOL”) implant, the occurrence of dysphotopsia can be remedied by analyzing the nasal retina and using information from the analysis in a corrective measure that includes implanting an intraocular lens (“IOL”) implant into an eye of a patient in need of measures to correct a dysphotopsia, behind an IOL.

Another unique aspect of the present teachings is the recognition that biometric information (e.g., nasal retina dimensional information) may be obtained using a noninvasive and, optionally, a non-contact technique. The biometric information can be used to predict the possibility of a positive or negative dysphotopsia in a patient in need of cataract removal surgery, to suggest and/or implement a prophylactic measure (e.g., prophylactic reverse optic capture, use of ciliary sulcus or capsular bag mask, or the like) to lower the possibility of a dysphotopsia (positive or negative) in a patient in need of cataract removal surgery, and/or to correct a positive or negative dysphotopsia in a patient in need of such a correction. Biometric information (e.g., nasal retina dimensional information) may be obtained using a noninvasive echo-location technique. Biometric information (e.g., nasal retina dimensional information) may be obtained using optical coherence tomography. Biometric information (e.g., nasal retina information) may be obtained using whole eye optical coherent tomography. Any combination of these procedures may be employed to obtain biometric information.

In use, according to elements of the teachings herein, the biometric information (e.g., nasal retina information) may be input into a computing system that includes a processor executing computer executable instructions programmed on a non-transitory storage medium in order to perform training of a computational model with a machine learning algorithm (e.g., artificial intelligence). The machine learning algorithm may include an artificial neural network (e.g., radial basis function artificial neural network). The computational model may, for example, account for the biometric information (e.g., nasal retina dimensional information). The biometric information (e.g., nasal retina dimensional information) may be analyzed in combination with other information.

By way of one illustration, it may be possible to use a noninvasive echo-location imaging technique to gather a plurality of images. The plurality of images may each depict a nasal retina of an eye of a different individual human or animal. Information embodied in the images representative of dimensions of the nasal retina may be input into a computational model. The computational model may be trained with the information. Information about a structure (e.g., a peripheral edge structure), material, location in eye, and/or dimensions (e.g., IOL optic size) of any implant (e.g., an intraocular lens implant or ophthalmic prosthetic) to be inserted into the eye may be input into the computational model. The computational model may be trained with this information. Demographic information about the patient may be input into the computational model. The computational model may be trained with this information. A personality profile of the patient may be input into the computational model. The computational model may be trained with this information. Data from studies (e.g., past or ongoing studies) about similar procedures performed upon different patients in need than the subject patient in need may be input into the computational model. The computational model may be trained with this information.

One of the beneficial results of the present teachings is the use, in particular, of whole eye optical coherent tomography prior to a cataract surgery upon a patient in need thereof. For example, the present teachings envision a process for reducing likelihood of dysphotopsia occurring as a result of implant lens surgery upon a patient in need of such implant lens surgery, comprising the steps of: (a) optionally obtaining auxiliary biometric information including one or any combination of: photographic measurements (e.g., two-dimensional photographs) of the eye upon which the surgery is to be performed; sectoral visual field information about the eye; and/or circumferential visual field information about the eye; (b) scanning the eye of a patient in need of the implant lens surgery, using a noninvasive echo-locating instrument, preferably an optical coherent tomographic instrument (e.g., a whole eye optical coherent tomographic instrument), for obtaining nasal retina information about the eye; and (c) based upon the optional auxiliary biometric information and the nasal retina dimensional information, employing at least one prophylactic measure for reducing likelihood of dysphotopsia as a result of implant lens surgery.

The teachings also envision a process for correcting dysphotopsia occurring as a result of intraocular lens implant surgery upon a patient in need of such correction, comprising the steps of: (a) optionally obtaining auxiliary biometric information including one or any combination of: photographic measurements (e.g., two-dimensional photographs) of the eye upon which the surgery is to be performed; sectoral visual field information about the eye; and/or circumferential visual field information about the eye; (b) scanning the eye of a patient in need of the intraocular lens implant surgery, using a noninvasive echo-locating instrument, preferably an optical coherent tomographic instrument (e.g., a whole eye optical coherent tomographic instrument), for obtaining nasal retina information about the eye; and (c) based upon the optional auxiliary biometric information and the nasal retina dimensional information, employing at least one corrective measure for reducing likelihood of dysphotopsia as a result of intraocular lens implant surgery.

### DETAILED DESCRIPTION

The present teachings derive from the recognition of various phenomena, heretofore either unrecognized or not fully understood. In particular, it has been determined that the ability to obtain and use valuable information about a portion of the eye known as the nasal retina can be a valuable tool in predicting the occurrence of positive or negative dysphotopsia before cataract removal surgery, and/or as a tool for correction of the occurrence of positive or negative dysphotopsia that has occurred after cataract removal surgery. Numerous benefits can be realized, including but not limited to improved overall surgery outcomes, improved patient comfort, and reduced visits by a patient to a medical facility.

Turning to one aspect of the present teachings, there is contemplated a process for reducing positive or negative dysphotopsia following intraocular lens implant surgery. The process may include a step of scanning the eye of a patient in need of the intraocular lens implant surgery, or correction of dysphotopsia (positive or negative), using a non-invasive echo-locating instrument, preferably a high-resolution ultrasound and/or an optical coherent tomographic instrument (e.g., a whole eye optical coherent tomographic instrument), for obtaining nasal retina information about the eye. Based at least in part from the nasal retina dimensional information, a prophylactic measure (e.g., prophylactic reverse optic capture, use of ciliary sulcus or capsular bag mask, or the like) can be recommended and/or implemented prior to cataract removal surgery, or a corrective measure can be recommended to address the dysphotopsia (positive or negative) after the cataract removal surgery. Use of artificial intelligence as part of the process is also contemplated.

In a more particularized aspect, it is possible that a noninvasive echo-locating instrument, preferably a high-resolution ultrasound and/or an optical coherence tomographic instrument (e.g., a whole eye optical coherence tomographic instrument) may be used alone or in combination with one or more imaging techniques (e.g., photography, preferably fundus photography) to obtain relevant information, preferably dimensional information, about the nasal retina for the subject eye. That relevant information can be employed to devise a prophylactic measure (e.g., a prophylactic reverse optic capture, use of ciliary sulcus or capsular bag mask, or the like) to reduce the likelihood of dysphotopsia prior to a cataract removal surgery, and/or a corrective measure to remediate an occurrence of dysphotopsia (positive or negative) after the cataract removal surgery (i.e., after the cataract has been removed and an intraocular lens implant has been implanted into the subject eye).

For situations in which likelihood of a dysphotopsia is to be reduced, (e.g., before cataract removal surgery and/or before implantation of an intraocular lens implant), a process for reducing likelihood of dysphotopsia occurring as a result of intraocular lens implant surgery upon a patient in need of such intraocular lens implant surgery is contemplated. The process may include optionally obtaining auxiliary biometric information from a patient in need of intraocular lens implant surgery. The auxiliary biometric information may include one or any combination of: photographic measurements (e.g., from two-dimensional photographs, such as may be obtained from fundus photography) of the eye upon which the surgery is to be performed; sectoral visual field information about the eye; circumferential visual field information about the eye. The process may include scanning the eye of a patient in need of the intraocular lens implant surgery, using a noninvasive echo-locating instrument, preferably an optical coherence tomographic instrument (e.g., a whole eye optical coherence tomographic instrument), for obtaining nasal retina information about the eye. Based upon the optional auxiliary biometric information and the nasal retina dimensional information, the process may employ at least one prophylactic measure (e.g., prophylactic reverse optic capture, use of ciliary sulcus or capsular bag mask, or the like) for reducing likelihood of dysphotopsia (positive or negative) as a result of intraocular lens implant surgery.

For situations in which a dysphotopsia (positive or negative) is to be corrected (e.g., after cataract removal surgery and/or after implantation of an intraocular lens implant), a process for correcting dysphotopsia occurring as a result of implant lens surgery upon a patient in need of such correction is contemplated. The process may include optionally obtaining auxiliary biometric information from a patient in need of intraocular lens implant surgery. The auxiliary biometric information may include one or any combination of: photographic measurements (e.g., from two-dimensional photographs, such as may be obtained from fundus photography) of the eye upon which the surgery has been performed; sectoral visual field information about the eye; circumferential visual field information about the eye. The process may include scanning the eye of a patient in need of the correction using a noninvasive echo-locating instrument, preferably an optical coherence tomographic instrument (e.g., a whole eye optical coherence tomographic instrument), for obtaining nasal retina information about the eye. Based upon the optional auxiliary biometric information and the nasal retina dimensional information, employing at least one corrective measure for reducing likelihood of dysphotopsia as a result of intraocular lens implant surgery.

Any of a number of additional considerations may apply to these processes. For example, the optical coherent tomographic instrument may be a handheld device.

The scanning may be performed at the location (e.g., the medical facility, such as a hospital, clinic, surgical center, doctor's office, or the like), of the intraocular lens implant surgery and before or during the same patient visit as the surgery.

The intraocular lens implant surgery may include removal of a cataract, which in turn may be followed by implantation of an intraocular lens implant.

The nasal retina information, for all or part of the nasal retina, may include nasal retina dimensions, geometry, and/or positional location. For example, the nasal retina information may include the anatomic location of some or all of the nasal retina, relative to one or more anterior segment structures of the eye.

At least one prophylactic measure for reducing likelihood of dysphotopsia may include inserting into the eye an intraocular lens implant having an edge structure. The edge structure may be dimensioned and configured for blocking passage of light through a gap located between an anterior surface of the intraocular implant lens and either or both of a posterior surface of an iris of the eye or an internal surface of a contiguous ciliary body of the eye.

At least one prophylactic measure for reducing likelihood of dysphotopsia may include placing an ophthalmic prosthetic (also referred to herein as a mask) within the capsular bag and/or ciliary sulcus as described in U.S. application Ser. No. 17/405,741 and International Application No. PCT/US2022/040315, incorporated by reference in their entirety. A combination of prophylactic measures disclosed herein may be employed.

At least one prophylactic measure for reducing likelihood of dysphotopsia, or measure to correct a dysphotopsia, may include inserting into the eye an ophthalmic prosthetic (such as that described in as described in U.S. application Ser. No. 17/405,741 and International Application No. PCT/US2022/040315, incorporated by reference in their entirety) dimensioned and configured for blocking passage of light through a gap located between an anterior surface of an intraocular implant lens in the eye, and either or both of a posterior surface of an iris of the eye or an internal surface of a contiguous ciliary body of the eye. A combination of prophylactic measures disclosed herein may be employed.

At least one prophylactic measure for reducing likelihood of dysphotopsia, or measure to correct a dysphotopsia, may include inserting into the eye an ophthalmic prosthetic (such as that described in as described in U.S. application Ser. No. 17/405,741 and International Application No. PCT/US2022/040315, incorporated by reference in their entirety) dimensioned and configured for blocking passage of light in a gap located between an anterior surface of an intraocular implant lens in the eye, and either or both of a posterior surface of an iris of the eye or an internal surface of a contiguous ciliary body of the eye. The ophthalmic prosthetic may be characterized by one or any combination of the following: (a) it is opaque, partially opaque, translucent, polarized, frosted, or any combination thereof; (b) a thickness of the ophthalmic prosthetic is from about 0.01 mm to about 1.2 mm, more preferably from about 0.05 mm to about 1 mm, or even more preferably from about 0.1 mm to about 0.5 mm; (c) the ophthalmic prosthetic has an annular sector, which optionally is configured to locate in the nasal hemisphere of an eye, temporal hemisphere of an eye, or both; (d) the ophthalmic prosthetic is annular and includes a centrally positioned though-hole opening; (e) the ophthalmic prosthetic has a centrally positioned though-hole opening with a diameter from about 1.5 mm to 6 mm, or even more preferably from about 2 mm to about 5 mm; (f) at least a portion of the ophthalmic prosthetic has been treated to modify a surface property of the prosthetic material (g) over at least a portion of the ophthalmic prosthetic it is flexible, foldable, compressible, dehydratable, rehydratable, or any combination thereof; (h) the ophthalmic prosthetic includes one or more haptics, which are configured to prevent the ophthalmic prosthetic from moving or rotating within an eye, and the one or more haptics are elongate projections that extend radially from an outer perimeter of the ophthalmic prosthetic. The process may include forming an incision in a cornea, inserting an ophthalmic prosthetic into an eye through the incision, and locating the ophthalmic prosthetic between the iris and an intraocular lens. The intraocular lens may be phakic or pseudophakic.

The present teachings contemplate that one or more additional procedures and associated devices can be used during the same patient visit as the surgery described herein. Non-limiting examples of such procedures may include those described in U.S. application Ser. No. 17/405,586 and International Application No. PCT/US2022/040287; as well as U.S. application Ser. No. 18/134,849; incorporated by reference in their entirety.

Any step of employing at least one prophylactic measure and/or corrective measure may be based upon a computer generated recommendation. The computer generated recommendation may be based upon a combination of nasal retina information about the eye and one or more of (a) a structure (e.g., a peripheral edge structure), material, location in eye, and/or dimensions of any implant (e.g., intraocular lens implant or ophthalmic prosthetic) to be inserted into the eye; (b) demographic information about the patient; (c) a personality profile of the patient; and (d) data from studies (e.g., past or ongoing studies) about similar procedures performed upon different patients in need than the subject patient in need. The computer-generated recommendation may be derived by a processor executing computer executable instructions programmed on a non-transitory storage medium to perform training of a computational model with a machine learning algorithm (e.g., artificial intelligence). For example, the machine learning algorithm may include an artificial neural network (e.g., a radial basis function artificial neural network).

The anterior edge of the anterior nasal retina may be identified physically by high resolution imaging such as high-resolution ultrasound or magnetic resonance imaging (“MRI”), though these modalities are impractical for screening purposes. Internal wide-field fundus photography may be one practical imaging technique that can identify the anterior retinal extent relative to anterior segment structures. This modality does not relate the anterior retinal edge to the anterior segment structures, though this may not be important, when combined with other biometry and functional data. The functional extent of the anterior edge of the nasal retina may be mapped by far-periphery visual field testing. This may be performed by some traditional visual field mechanisms, virtual reality visual field devices, or even one or more meridian simulations (much like the peripheral visual field threshold testing performed at many States' Bureau of Motor Vehicles). This surrogate for the location of the anterior retinal extent could be obviated by direct measures of the anterior retina and anterior segment structures simultaneously using the new technology of whole eye optical coherence tomography.

Other considerations may be a part of the teachings described herein. For example, auxiliary biometric information may be used alone, in combination with 2-dimensional photographic information (without any “whole eye” optical coherence tomography), in combination with “whole eye” optical coherence tomography (but no 2-dimensional photographic information), or in combination with echo-locating imaging (e.g., whole eye optical coherence tomography) and 2-dimensional photographic information.

The process may include a step of assessing a risk of dysphotopsia (positive or negative) based upon information about anatomic relationships of a particular patient. The anatomic relationship information may include either or both of anterior segment and posterior segment microanatomy. The anatomic relationship information may include information about a relative orientation of a globe in the orbit in primary gaze of a patient.

The information may be employed, in accordance with the present teachings, to predict an outcome for a patient. For example, the information may be employed to predict, for a patient (e.g., before a cataract removal surgery and/or before a dysphotopsia corrective measure is taken) whether and to what extent 1) the intraocular lens implant edge will be accessible to ambient light, and/or 2) the relative anterior limit of the neurosensory retina ability to capture such light that may either be diverted by the intraocular lens implant edge or pass beyond the intraocular lens implant edge through the peripheral aphakic space.

The present teachings may employ biometric information as a basis, such as through the use of artificial intelligence (“Al”), to determine a dysphotopsia prophylactic measure and/or a corrective measure. The biometric information may include one or any combination of measurements selected from corneal dimension (e.g., curvature and/or width); axial length (i.e., anterior chamber depth, lens thickness, and vitreous chamber depth), anterior chamber depth (“ACD”), chord mu (i.e., a two-dimensional distance between a pupil center (“line of sight”) and a subject-fixated coaxially sighted corneal light reflex); angle kappa (i.e., an angle between a visual axis (a line connecting a fixation point with a fovea) and the pupillary axis (a line that perpendicularly passes through the entrance pupil and a cornea curvature center)); one or more patient pre-surgical lens and/or intraocular lens implant characteristics (e.g., dimension such as diameter, thickness, and curvature; power, any decentration information; or other information); one or more nasal retina characteristics (e.g., dimension such as diameter, thickness, and curvature; location; or other information); or a Goldmann kinetic visual field measurement.

Thus, it is seen that the present teachings may envision use of multivariate measurements of a patient in need. In this manner, it may be possible to take into account one or more interactions between each factor in the physical microanatomy (e.g., including corneal curvatures) and the functional or anatomic anterior retinal extent. Thus, decision-making may be adjusted based on the multivariate measurements.

Accordingly, the present teachings envision the collection and storage on a non-transitory storage medium of information about an individual patient and/or a group patients. The data may include searchable and/or sortable data about biometric information, patient personality profile information, patient demographic information, and outcome prediction information. The data may be anonymized The database may include historic information about eyes that have been diagnosed with negative and/or positive dysphotopsia.

It will be appreciated, accordingly, that artificial intelligence techniques, such as radial basis function, can be used to predict which factors are the most important and in what combination they are most likely to result in symptomatic complaints. Additional testing and risk stratification can be incorporated into the pre-operative cataract “work-up.” Risk stratification can guide intraocular lens implant choice, possible prophylactic measures (e.g., placement of a ciliary sulcus or capsular bag “mask”), and patient counselling, both for realistic expectation, and, perhaps, for the patient to decide whether to delay or defer intervention if they are in a high risk category.

To summarize, some of the various aspects of the present teachings, in general, envision applying an algorithm to a database of eye biometry and functional visual data for the purpose of dysphotopsia risk stratification. The algorithm may include simple regression, artificial intelligence analysis, or any combination or variation thereof. The relative accuracy of the algorithm will increase as additional data is added. Thus, the process becomes a “living system” which can adapt to increasing and/or changing data and/or parameters. Different intraocular lens implant models, thicknesses, and/or biomaterials may improve the predictive value of the system. Data collected from the intraocular lens implant information within the database may potentially be used to aid in intraocular lens implant designs.

Biometric information (e.g., nasal retina information) may be obtained using a noninvasive and optionally a non-contact technique. Biometric information (e.g., nasal retina information) may be obtained using a noninvasive echo-location technique. Biometric information (e.g., nasal retina information) may be obtained using one or more optical tomographic techniques. For example, such information may be obtained using optical coherence tomography (e.g., partial or whole eye tomography), such as with a suitable imaging instrument. The optical coherence tomography imaging instrument may be, but need not necessarily be, a whole eye optical coherence tomography imaging instrument, such that whole eye optical coherence tomography is performed.

Optical coherence tomography generally, and illustrative examples of optical coherence tomography imaging instruments (and methods of using thereof), which can be employed in the present teachings are discussed in U.S. Pat. Nos. 9,649,027 B2 and 10,694,939 B2, both hereby incorporated by reference in their entirety. U.S. Pat. No. 10,694,939 B2, in particular, indicates the suitability of its teachings for whole eye optical coherence tomography.

It is envisioned that the techniques of the present teachings may also employ artificial intelligence in providing a recommended prophylactic measure to help improve chances of a successful procedure. The present teachings also may be employed to aid in predicting the outcome of an ophthalmological procedure.

The use of artificial intelligence with optical coherence tomography is illustrated in U.S. Patent Application Nos. 2020/0077883 A1; 2021/0295508 A1; and 2020/0245858 A1; each hereby incorporated by reference in their entirety.

By way of one illustration, it may be possible to use a non-invasive echo-location imaging technique to gather a plurality of images. Each image may depict a nasal retina of an eye of a different individual human or animal. Information embodied in the images that is representative of the dimensions of the nasal retina may be inputted into a computational model. The computational model may be trained with the information.

Information about a structure (e.g., a peripheral edge structure), material, location in the eye, and/or dimensions (e.g., intraocular lens optic size) of any implant (e.g., intraocular lens implant or ophthalmic prosthetic) to be inserted into the eye may be inputted into the computational model. The computational model may be trained with this information.

Demographic information about the patient may be inputted into the computational model. The computational model may be trained with this information.

A personality profile of the patient may be inputted into the computational model. The computational model may be trained with this information.

Data from studies (e.g., past or ongoing studies) about same or similar procedures performed upon different patients in need than the subject patient in need may be inputted into the computational model. The computational model may be trained with this information.

Systems in accordance with the present teachings may include one or processors, one or more non-transitory storage media, one or more image capture devices (e.g., a camera, a tomography imaging device or otherwise), optionally one or more ophthalmological surgical instruments, one or more graphical display devices, or any combination thereof. The one or more non-transitory storage media (e.g., hard disk drive, solid state drive, memory card, disk drive, compact disk (CD), or electronic storage on a server accessible over a network) may store program instructions.

Any of the techniques herein, whether for gathering biometric information (e.g., nasal retina information), may be robotically assisted. For example, one or more of the image capture devices (e.g., a camera, a tomography imaging device, or otherwise), or the one or more of the ophthalmological surgical instruments may be carried on a robot arm. The robot arm may be controlled directly by a human operator. The robot arm may be configured to receive instructions from an electronic processor. The instructions may be derived from the execution of machine learning.

The present teachings may be useful in a predictive pre-operational scenario, such as prior to performance of cataract surgery. The present teachings can also be used in a post-operational scenario in which, following a cataract removal procedure, a patient presents indications of dysphotopsia (positive or negative) requiring a corrective measure to be taken. Thus, it may be possible that an intraocular lens implant may already be present, or an intraocular lens implant may not already be present in an eye of the patient at the time the present teachings are employed.

### Example 1

A patient in need of cataract removal surgery is examined prior to surgery. The examination includes a step of acquiring biometric information. The step of acquiring biometric information includes a step of performing measurements of one or more dimensional characteristics of the patient eye. In addition, wide field fundus photography is performed, as is whole eye optical coherence tomography, for acquiring information about the nasal retina. Based at least in part upon the acquired information about the nasal retina, a prophylactic measure to reduce the likelihood of dysphotopsia (positive or negative) is performed during the cataract removal surgery. Following surgery, the patient exhibits no indication of dysphotopsia.

### Example 2

The same procedure as Example 1 is performed. However, instead of whole eye optical coherence tomography, another echo-location technique is used (namely ultra-high resolution ultrasound), and a similar outcome is realized.

### Examples 3a and 3b

The same procedures as Examples 1 and 2 are performed. In addition to the measurements, information is also acquired about peripheral visual field extent, and a similar outcome is realized.

### Example 4

The same procedures as Examples 1, 2, 3a, and 3b are performed, except that determination of the prophylactic measure is guided by the use of the acquired information being used by a processor executing computer executable instructions programmed on a non-transitory storage medium to perform training of a computational model with a machine learning algorithm (e.g., artificial intelligence), and particularly the machine learning algorithm includes an artificial neural network (e.g., a radial basis function artificial neural network). Also, all of the acquired information is entered into a database along with the outcome of the surgery, so that it can be used for guiding future surgical procedures for other patients in need. A similar successful outcome is realized.

### Example 5

The same procedure as Example 4 is performed, except the acquired information also contains information about the patient demographics and/or personality.

The following are general teachings applicable to all embodiments.

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The terms “generally” or “substantially” to describe angular measurements may mean about +/−10° or less, about +/−5° or less, or even about +/−1° or less.

The terms “generally” or “substantially” to describe angular measurements may mean about +/−0.01° or greater, about +/−0.1° or greater, or even about +/−0.5° or greater.

The terms “generally” or “substantially” to describe linear measurements, percentages, or ratios may mean about +/−10% or less, about +/−5% or less, or even about +/−1% or less.

The terms “generally” or “substantially” to describe linear measurements, percentages, or ratios may mean about +/−0.01% or greater, about +/−0.1% or greater, or even about +/−0.5% or greater.

Unless otherwise stated, any numerical values recited herein include all values from the lower value to the upper value in increments of one unit, provided that there is a separation of at least 2 units between any lower value and any higher value. As an example, if it is stated that the amount of a component, a property, or a value of a process variable such as, for example, temperature, pressure, time, and the like is, for example, from 1 to 90, from 20 to 80, or from 30 to 70, it is intended that intermediate range values such as (for example, 15 to 85, 22 to 68, 43 to 51, 30 to 32 etc.) are within the teachings of this specification. Likewise, individual intermediate values are also within the present teachings. For values which are less than one, one unit is considered to be 0.0001, 0.001, 0.01, or 0.1 as appropriate. These are only examples of what is specifically intended and all possible combinations of numerical values between the lowest value and the highest value enumerated are to be considered to be expressly stated in this application in a similar manner

Unless otherwise stated, all ranges include both endpoints and all numbers between the endpoints.

The term “consisting essentially of” to describe a combination shall include the elements, ingredients, components, or steps identified, and such other elements ingredients, components, or steps that do not materially affect the basic and novel characteristics of the combination. The use of the terms “comprising” or “including” to describe combinations of elements, ingredients, components, or steps herein also contemplates embodiments that consist essentially of the elements, ingredients, components, or steps.

Plural elements, ingredients, components, or steps can be provided by a single integrated element, ingredient, component, or step. Alternatively, a single integrated element, ingredient, component, or step might be divided into separate plural elements, ingredients, components, or steps. The disclosure of “a” or “one” to describe an element, ingredient, component, or step is not intended to foreclose additional elements, ingredients, components, or steps.

It is understood that the above description is intended to be illustrative and not restrictive. Many embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled.

The disclosures of all articles and references, including patent applications and publications, are incorporated by reference in their entirety for all purposes.

What is claimed is:

1: A process for reducing a likelihood of dysphotopsia occurring as a result of intraocular lens implant surgery upon a patient in need of such intraocular lens implant surgery, the process comprising the steps of:
a) optionally obtaining auxiliary biometric information from the patient in need of the intraocular lens implant surgery, including one or any combination of:
i) photographic measurements, optionally from two-dimensional photographs obtained from fundus photography of an eye upon which the intraocular lens implant surgery is to be performed;
ii) sectoral visual field information about the eye; and
iii) circumferential visual field information about the eye;
b) scanning the eye of the patient in need of the intraocular lens implant surgery, using a noninvasive echo-locating instrument, optionally an optical coherence tomographic instrument including a whole eye optical coherence tomographic instrument, for obtaining nasal retina information about the eye, optionally the nasal retina information including, for all or part of a nasal retina, its dimensions, geometry, and/or positional location; and
c) based upon the optional auxiliary biometric information and the nasal retina information, employing at least one prophylactic measure for reducing the likelihood of dysphotopsia as a result of the intraocular lens implant surgery.

2: The process of claim 1, wherein the optical coherence tomographic instrument is a handheld device, and the scanning is performed at a location of the intraocular lens implant surgery and during a same patient visit as the intraocular lens implant surgery.

3: The process of claim 2, wherein the intraocular lens implant surgery includes removal of a cataract.

4: The process of claim 3, wherein the nasal retina information includes an anatomic location of the nasal retina, relative to one or more anterior segment structures.

5: The process of claim 4, wherein the at least one prophylactic measure for reducing the likelihood of dysphotopsia includes inserting into the eye an intraocular lens implant having an edge structure dimensioned and configured for blocking passage of light in a gap located between an anterior surface of the intraocular lens implant and either or both of a posterior surface of an iris of the eye or an internal surface of a contiguous ciliary body of the eye; and/or placing a mask within a capsular bag and/or a ciliary sulcus of the eye.

6: The process of claim 4, wherein the at least one prophylactic measure for reducing the likelihood of dysphotopsia includes inserting into the eye an ophthalmic prosthetic dimensioned and configured for blocking passage of light in a gap located between an anterior surface of the intraocular lens implant and either or both of a posterior surface of an iris of the eye or an internal surface of a contiguous ciliary body of the eye.

7: The process of claim 4, wherein the at least one prophylactic measure for reducing the likelihood of dysphotopsia includes inserting into the eye an ophthalmic prosthetic dimensioned and configured for blocking passage of light in a gap located between an anterior surface of the intraocular implant lens, and either or both of a posterior surface of an iris of the eye or an internal surface of a contiguous ciliary body of the eye, and the ophthalmic prosthetic is characterized by one or any combination of the following: (a) it is opaque, partially opaque, translucent, polarized, frosted, or any combination thereof; (b) a thickness of the ophthalmic prosthetic is from about 0.01 mm to about 1.2 mm; (c) the ophthalmic prosthetic has an annular sector, which optionally is configured to locate in a nasal hemisphere of the eye, a temporal hemisphere of the eye, or both; (d) the ophthalmic prosthetic is annular and includes a centrally positioned though-hole opening; (e) the ophthalmic prosthetic has a centrally positioned though-hole opening with a diameter of about 1.5 mm to 6 mm; (f) at least a portion of the ophthalmic prosthetic has been treated to modify a surface property of the ophthalmic prosthetic (g) over at least a portion of the ophthalmic prosthetic it is flexible, foldable, compressible, dehydratable, rehydratable, or any combination thereof; (h) the ophthalmic prosthetic includes one or more haptics, which are configured to prevent the ophthalmic prosthetic from moving or rotating within the eye, and the one or more haptics are elongate projections that extend radially from an outer perimeter of the ophthalmic prosthetic.

8: The process of claim 7, wherein the process includes a step of forming an incision in a cornea of the eye, inserting the ophthalmic prosthetic into the eye through the incision, and locating the ophthalmic prosthetic between the iris and an intraocular lens implant.

9: The process of claim 1, wherein the step of employing the at least one prophylactic measure is based upon a computer generated recommendation that is based upon a combination of the nasal retina information about the eye and one or more of (a) a structure (optionally a peripheral edge structure), material, location in eye, and/or dimensions, of any implant including an intraocular lens or an ophthalmic prosthetic to be inserted into the eye; (b) demographic information about the patient; (c) a personality profile of the patient; and (d) data from past or ongoing studies about similar procedures performed upon different patients in need than the patient in need.

10: The process of claim 9, wherein the computer-generated recommendation is derived by a processor executing computer executable instructions programmed on a non-transitory storage medium to perform training of a computational model with a machine learning algorithm.

11: The process of claim 10, wherein the machine learning algorithm includes an artificial neural network, optionally a radial basis function artificial neural network.

12: A process for correcting dysphotopsia occurring as a result of intraocular lens implant surgery upon a patient in need of such intraocular lens implant surgery, the process comprising the steps of:
a) optionally obtaining auxiliary biometric information from the patient in need of the intraocular lens implant surgery, including one or any combination of:
i) photographic measurements, optionally from two-dimensional photographs obtained from fundus photography of an eye upon which the intraocular lens implant surgery is to be performed;
ii) sectoral visual field information about the eye; and
iii) circumferential visual field information about the eye;
b) scanning the eye of the patient in need of the intraocular lens implant surgery, using a noninvasive echo-locating instrument, optionally an optical coherent tomographic instrument including a whole eye optical coherent tomographic instrument, for obtaining nasal retina information about the eye, optionally the nasal retina information including, for all or part of a nasal retina, its dimensions, geometry, and/or positional location; and
c) based upon the optional auxiliary biometric information and the nasal retina information, employing at least one corrective measure for correcting dysphotopsia as a result of the intraocular lens implant surgery.

13: The process of claim 12, wherein the optical coherence tomographic instrument is a handheld device, and the scanning is performed at a location of the intraocular lens implant surgery and during a same patient visit as the intraocular lens implant surgery.

14: The process of claim 13, wherein the intraocular lens implant surgery includes removal of a cataract.

15: The process of claim 14, wherein the nasal retina information includes an anatomic location of the nasal retina, relative to one or more anterior segment structures.

16: The process of claim 15, wherein the at least one corrective measure for reducing the likelihood of dysphotopsia includes inserting into the eye an intraocular lens implant having an edge structure dimensioned and configured for blocking passage of light in a gap located between an anterior surface of the intraocular lens implant and either or both of a posterior surface of an iris of the eye or an internal surface of a contiguous ciliary body of the eye; and/or placing a mask within a capsular bag and/or a ciliary sulcus of the eye.

17: The process of claim 15, wherein the at least one corrective measure for reducing the likelihood of dysphotopsia includes inserting into the eye an ophthalmic prosthetic dimensioned and configured for blocking passage of light in a gap located between an anterior surface of the intraocular lens implant and either or both of a posterior surface of an iris of the eye or an internal surface of a contiguous ciliary body of the eye.

18: The process of claim 15, wherein the at least one corrective measure for reducing the likelihood of dysphotopsia includes inserting into the eye an ophthalmic prosthetic dimensioned and configured for blocking passage of light in a gap located between an anterior surface of the intraocular implant lens, and either or both of a posterior surface of an iris of the eye or an internal surface of a contiguous ciliary body of the eye, and the ophthalmic prosthetic is characterized by one or any combination of the following: (a) it is opaque, partially opaque, translucent, polarized, frosted, or any combination thereof; (b) a thickness of the ophthalmic prosthetic is from about 0.01 mm to about 1.2 mm; (c) the ophthalmic prosthetic has an annular sector, which optionally is configured to locate in a nasal hemisphere of the eye, a temporal hemisphere of the eye, or both; (d) the ophthalmic prosthetic is annular and includes a centrally positioned though-hole opening; (e) the ophthalmic prosthetic has a centrally positioned though-hole opening with a diameter of about 1.5 mm to 6 mm; (f) at least a portion of the ophthalmic prosthetic has been treated to modify a surface property of the ophthalmic prosthetic (g) over at least a portion of the ophthalmic prosthetic it is flexible, foldable, compressible, dehydratable, rehydratable, or any combination thereof; (h) the ophthalmic prosthetic includes one or more haptics, which are configured to prevent the ophthalmic prosthetic from moving or rotating within the eye, and the one or more haptics are elongate projections that extend radially from an outer perimeter of the ophthalmic prosthetic.

19: The process of claim 18, wherein the process includes a step of forming an incision in a cornea of the eye, inserting the ophthalmic prosthetic into the eye through the incision, and locating the ophthalmic prosthetic between the iris and an intraocular lens implant.

20: The process of claim 1, wherein the step of employing the at least one corrective measure is based upon a computer generated recommendation that is based upon a combination of the nasal retina information about the eye and one or more of (a) a structure (optionally a peripheral edge structure), material, location in eye, and/or dimensions, of any implant including an intraocular lens or an ophthalmic prosthetic to be inserted into the eye; (b) demographic information about the patient; (c) a personality profile of the patient; and (d) data from past or ongoing studies about similar procedures performed upon different patients in need than the patient in need; optionally wherein the computer-generated recommendation is derived by a processor executing computer executable instructions programmed on a non-transitory storage medium to perform training of a computational model with a machine learning algorithm; optionally wherein the machine learning algorithm includes an artificial neural network; and optionally wherein the artificial neural network includes a radial basis function artificial neural network.
